SEQUENCE LISTING (32 sequences)

SEQ ID NO: 1 - protein, 7 aa, Kluyvermyces lactis
QGYNAQQ

SEQ ID NO: 2 - protein, 11 aa, Yarrowia lipolytica
GGALKIGGDKP

SEQ ID NO: 3 - DNA, 30 bp, Artificial Sequence
  Description of Artificial Sequence PCR primer
ccggatccatatgtctaaccctcaagatca

SEQ ID NO: 4 - DNA, 30 bp, Artificial Sequence
  Description of Artificial Sequence PCR primer
ccggatccatatgtctgacgatcaacagta

SEQ ID NO: 5 - DNA, 30 bp, Artificial Sequence
  Description of Artificial Sequence PCR primer
ccggatccatatgtcagaccaacaaaatca

SEQ ID NO: 6 - DNA, 30 bp, Artificial Sequence
  Description of Artificial Sequence PCR primer
ccggatccatatgagtgatcaattcaacca

SEQ ID NO: 7 - DNA, 30 bp, Artificial Sequence
  Description of Artificial Sequence PCR primer
ccggatccatatgtctgacccaaaccagaa

SEQ ID NO: 8 - DNA, 29 bp, Artificial Sequence
  Description of Artificial Sequence PCR primer
ggggatccaatgtcagaccaacaaaatca

SEQ ID NO: 9 - DNA, 30 bp, Artificial Sequence
  Description of Artificial Sequence PCR primer
gggctgcaggagcaccttgttggccgttgt

SEQ ID NO: 10 - DNA, 29 bp, Artificial Sequence
  Description of Artificial Sequence PCR primer
ggggatccaatgtcagaccaacaaaatca

SEQ ID NO: 11 - DNA, 30 bp, Artificial Sequence
  Description of Artificial Sequence PCR primer
ggggatatccggttggctgttgtgcattat

SEQ ID NO: 12 - DNA, 30 bp, Artificial Sequence
  Description of Artificial Sequence PCR primer
gggctgcaggctaccaagcatatcaagctt

SEQ ID NO: 13 - DNA, 30 bp, Artificial Sequence; identical to SEQ ID NO: 9
  Description of Artificial Sequence PCR primer

SEQ ID NO: 14 - DNA, 30 bp, Artificial Sequence
  Description of Artificial Sequence PCR primer
gggatatcaagctccagcacagtcttcatc

SEQ ID NO: 15 - DNA, 30 bp, Artificial Sequence
  Description of Artificial Sequence PCR primer
gggctcgagttaattttcaaggattttcac

SEQ ID NO: 16 - DNA, 30 bp, Artificial Sequence
  Description of Artificial Sequence PCR primer
ggggtcgacatggtgagcaagggcgaggag

SEQ ID NO: 17 - DNA, 29 bp, Artificial Sequence
  Description of Artificial Sequence PCR primer
ggggagctcttacttgtacagctcgtcca

SEQ ID NO: 18 - DNA, 30 bp, Artificial Sequence; identical to SEQ ID NO: 5
  Description of Artificial Sequence PCR primer

SEQ ID NO: 19 - DNA, 30 bp, Artificial Sequence
  Description of Artificial Sequence PCR primer
gggggtcgacatctttaacgacttcttcgt

SEQ ID NO: 20 - DNA, 30 bp, Artificial Sequence; identical to SEQ ID NO: 4
  Description of Artificial Sequence PCR primer

SEQ ID NO: 21 - DNA, 30 bp, Artificial Sequence
  Description of Artificial Sequence PCR primer
gggggtcgacatccttgacaacttcttcat

SEQ ID NO: 22 - DNA, 30 bp, Artificial Sequence
  Description of Artificial Sequence PCR primer
ggggtcgacatggctagcaaaggagaagaa

SEQ ID NO: 23 - DNA, 24 bp, Artificial Sequence
  Description of Artificial Sequence PCR primer
ggggagctcgatccttatttgtat

SEQ ID NO: 24 - DNA, 29 bp, Artificial Sequence
  Description of Artificial Sequence PCR primer
ggggatccaatgtcagaccaacaaaatca

SEQ ID NO: 25 - DNA, 30 bp, Artificial Sequence
  Description of Artificial Sequence PCR primer
gggaggccttacccacttcaatggttttac

SEQ ID NO: 26 - DNA, 30 bp, Artificial Sequence
  Description of Artificial Sequence PCR primer
gggctgcagctctcaacaagctcaagaagc

SEQ ID NO: 27 - DNA, 30 bp, Artificial Sequence
  Description of Artificial Sequence PCR primer
ggggatatccctccttcttctcgctctcct

SEQ ID NO: 28 - DNA, 142 bp, Artificial Sequence
  Description of Artificial Sequence SYNTHETIC DNA
gggctgcaggccagcaacaacagcagcagcagcaacaacagcaacaacagcaacagcaacaacaacagcaacagcaacagcagcagcaacagcaacaacagcaacagcaacagcagcaacaacaacaatacggatatccccc

SEQ ID NO: 29 - DNA, 15 bp, Artificial Sequence
  Description of Artificial Sequence PCR primer
gggctgcaggccagc

SEQ ID NO: 30 - DNA, 15 bp, Artificial Sequence
  Description of Artificial Sequence PCR primer
gggggatatccgtat

SEQ ID NO: 31 - DNA, 30 bp, Artificial Sequence; identical to SEQ ID NO: 7
  Description of Artificial Sequence PCR primer

SEQ ID NO: 32 - DNA, 30 bp, Artificial Sequence
  Description of Artificial Sequence PCR primer
gggggtcgacatcattaacgaccccttcat

(19) United States
(12) Patent Application Publication
(10) Pub. No.: US 2004/0158040 A1
(43) Pub. Date: Aug. 12, 2004

(54) Peptides participating in prionization of heterogenous erf3

(76) Inventors: Yoshikazu Nakamura, Tokyo (JP); Toru Nakayashiki, Tokyo (JP)

Correspondence Address: DARBY & DARBY P.C., P. O. BOX 5257, NEW YORK, NY, 10150-5257, US

(21) Appl. No.: 10/481,763

(22) PCT Filed: May 24, 2002
(86) PCT No.: PCT/JP02/05031

(30) Foreign Application Priority Data
Jun. 19, 2001 (JP) 2001-185039

(51) Int. Cl. C07K 14/39; C12Q 1/70; C07H 21/04; C12N 1/18

(52) U.S. Cl. 530/350; 435/69.1; 435/254.2; 435/320.1; 536/23.2

(57) ABSTRACT

The present invention provides eRF3 proteins capable of prionizing heterologous eRF3 proteins, and eRF3 proteins capable of being prionized by heterologous eRF3 proteins. Using these proteins, screening for substances that inhibit transmission of abnormal PrP, detection of abnormal PrP, creation of abnormal PrP, and screening for substances that restore abnormal PrP to normal PrP are carried out.

### TECHNICAL FIELD

The present invention relates to peptides involved in the transmission of prionization between heterologous eRF3 proteins, as well as the following methods using such peptides: a method of screening for substances that inhibit transmission of abnormal PrP, a method of detecting abnormal PrP, a method of creating abnormal PrP, and a method for screening substances that restore abnormal PrP to normal PrP.

### BACKGROUND ART

It is believed that mammalian prion diseases, such as Creutzfeldt-Jakob disease (CDJ), kuru, scrapie and bovine spongiform encephalopathy (BSE), are transmitted by a protein called PrP per se. In prion, a single gene product can generate two proteins with different conformations; the pathogenic abnormal protein (PrP^{sc}) has a nature that it can convert the normal protein (PrP^{c}) into the abnormal form. A protein having such a nature has also been reported in yeast, and is called yeast prion. Although yeast prion has no relation with PrP in its function as a protein, it has much in common with PrP in terms of prionic characteristics. For example, the following characteristics 1) to 5) are common to yeast prion and PrP. 1) They do not follow Mendelian genetic laws (cytoplasmic genes), and wild-type gene products exhibit abnormal natures without mutations in their genes. 2) Excessive expression of these proteins enhances prionization. 3) Abnormal proteins form fiber. Furthermore, in eRF3, which is one of yeast prions, 4) repetitions of an oligopeptide motif (eRF3: PQGGYQQYN, PrP: PHGGGWGQ) are present at the N-terminal of the protein, and the number of these repetitions influences upon frequency of prionization. 5) A species barrier for transmission exists depending on species.

Considering these common characteristics as prion proteins, it is highly possible that conformational changes in yeast eRF3 and mammalian PrP are caused by a common mechanism. Thus, it is believed that the mechanism of conformational changes in PrP can be elucidated by closely examining the processes of prionization in yeast eRF3.

The present invention has been made under the above-described technical background. It is an object of the invention to specify those sites in yeast eRF3 that are involved in the transmission of prionization and to elucidate the mechanism of the transmission of prionization.

### DISCLOSURE OF THE INVENTION

As a result of intensive and extensive researches toward the solution of the above problems, the present inventors have found several sequences that play an important role in the transmission of prionization between heterologous eRF3 proteins. Furthermore, the inventors have found that the transmission of prionization mediated via one of those sequences resembles the transmission of abnormal PrP in many points. The present invention has been completed based on these findings.

The first aspect of the present invention relates to a peptide represented by the amino acid sequence as shown in SEQ ID NO: 1.

The second aspect of the present invention relates to a yeast eRF3 in which repetitions of the amino acid sequence as shown in SEQ ID NO: 1 are integrated into its prion domain.

The third aspect of the present invention relates to a peptide represented by the amino acid sequence as shown in SEQ ID NO: 2.

The fourth aspect of the present invention relates to a yeast eRF3 in which repetitions of the amino acid sequence as shown in SEQ ID NO: 2 are integrated into its prion domain.

The fifth aspect of the present invention relates to a yeast eRF3 in which a part of the sequence of its prion domain is replaced with a polyglutamine sequence.

The sixth aspect of the present invention relates to a method of screening for substances that inhibit transmission of an abnormal PrP, comprising the following steps (1) to (3):

(1) contacting a yeast having a prionized eRF3 with a test substance,

(2) allowing the yeast in step (1) to express a yeast eRF3 having repetitions of the amino acid sequence as shown in SEQ ID NO: 2 in its prion domain, and

(3) determining whether or not the test substance inhibits prionization of the yeast eRF3 expressed in step (2).

The seventh aspect of the present invention relates to a method of detecting an abnormal PrP, comprising the following steps (1) and (2):

(1) transferring a test sample into cells of a yeast comprising a yeast eRF3 in which repetitions of the amino acid sequence as shown in SEQ ID NO: 2 or a PrP sequence is integrated into its prion domain, and

(2) determining whether the yeast eRF3 is prionized or not.

The eighth aspect of the present invention relates to a method of creating an abnormal PrP, comprising contacting a normal PrP with a yeast eRF3 in which repetitions of the amino acid sequence as shown in SEQ ID NO: 1 are integrated into its prion domain or a yeast eRF3 in which a part of the sequence of its prion domain is replaced with a polyglutamine sequence.

The ninth aspect of the present invention relates to a method of screening for substances that restore an abnormal PrP to a normal PrP, comprising the following steps (1) to (3):

(1) obtaining an abnormal PrP by contacting a normal PrP with a yeast eRF3 in which repetitions of the amino acid sequence as shown in SEQ ID NO: 1 are integrated into its prion domain or a yeast eRF3 in which a part of the sequence of its prion domain is replaced with a polyglutamine sequence,

(2) contacting the abnormal PrP with a test substance, and

(3) determining whether or not the test substance restores the abnormal PrP to a normal PrP.

Hereinbelow, the present invention will be described in detail.

(A) Kluyvermyces lactis-Derived Peptide

The peptide represented by the amino acid sequence as shown in SEQ ID NO: 1 (Gln Gly Tyr Asn Ala Gln Gln) is a peptide contained in the prion domain of Kluyvermyces lactis-derived eRF3. This peptide has the functions as described below. A prionized eRF3 acts to prionize normal eRF3 proteins, but such transmission of prionization only occurs between eRF3 proteins of the same species and not between heterologous eRF3 proteins. However, when an eRF3 in which repetitions of the sequence of this peptide are integrated in its prion domain is used as a source of transmission, prionization is transmitted not only between eRF3 proteins of the same species but also between heterologous eRF3 proteins. Therefore, this peptide or an eRF3 in which repetitions of the sequence of this peptide are integrated into its prion domain can be used in the preparation of models for investigating the mechanism of transmission of prionization between heterologous eRF3 proteins. Furthermore, such models can be used in the screening for substances that inhibit the transmission of prionization between heterologous eRF3 proteins.

The amino acid sequence of the K. lactis-derived eRF3 is registered at the GenBank (Accession Number: AB039749).

(B) Yarrowia lipolytica-Derived Peptide

The peptide represented by the amino acid sequence as shown in SEQ ID NO: 2 (Gly Gly Ala Leu Lys Ile Gly Gly Asp Lys Pro) is a peptide contained in the prion domain of Y lipolytica-derived eRF3. This peptide has the functions as described below. The transmission of prionization only occurs, as described above, between eRF3 proteins of the same species in principle. However, when an eRF3 in which repetitions of the sequence of this peptide are integrated in its prion domain is used as a normal eRF3 to be prionized, transmission of prionization occurs between heterologous eRF3 proteins. Therefore, this peptide or an eRF3 in which repetitions of the sequence of this peptide are integrated into its prion domain can also be used in the preparation of models for investigating the mechanism of transmission of prionization between heterologous eRF3 proteins. The manner in which prionization is transmitted via this peptide is different in many points from the manner in which prionization of other eRF3 proteins derived from, e.g., K. lactis is transmitted, and rather resembles the transmission of abnormal PrP. Therefore, this peptide can also be used in a method of screening for substances that inhibit transmission of abnormal PrP, as described later.

The amino acid sequence of the Y. lipolytica-derived eRF3 is registered at the BenBank (Accession Number: AB039752).

(C) Method of Screening for Substances that Inhibit Transmission of Abnormal PrP

The transmission of prionization of yeast eRF3 via repetitions of the amino acid sequence as shown in SEQ ID NO: 2 is different from the transmission of prionization of other eRF3 proteins in the points described below, and resembles the transmission of abnormal PrP.

(i) Repeat motifs present in the prion domain of yeast eRF3 proteins contain a number of Asn and Gln. This characteristic is not observed in repeat motifs of PrP. The amino acid sequence as shown in SEQ ID NO: 2 contains neither Asn nor Gln, and thus resembles repeat motifs of PrP.

(ii) Prionized eRF3 proteins are restored to normal eRF3 proteins with guanidine hydrochloride, but this nature has not been confirmed in PrP. When an eRF3 comprising repetitions of the amino acid sequence as shown in SEQ ID NO: 2 has been prionized, this protein is not restored with guanidine hydrochloride. It also resembles PrP in this point.

(iii) Prionized eRF3 proteins are restored to normal eRF3 proteins by disruption of HSP104 gene, but this nature has not been confirmed in PrP. When an eRF3 comprising repetitions of the amino acid sequence as shown in SEQ ID NO: 2 has been prionized, this protein is not restored by disruption of HSP104 gene. It also resembles PrP in this point.

Considering what have been described above, it is highly possible that the transmission of eRF3 prionization via the amino acid sequence as shown in SEQ ID NO: 2 is caused by a mechanism that is similar to the mechanism causing the transmission of abnormal PrP. Thus, it is believed that it can be possible to screen for substances that inhibit transmission of with abnormal PrP by screening for substances that inhibit transmission of prionized eRF3 using the following steps (1) to (3).

(1) A step of contacting a yeast having a prionized eRF3 with a test substance

(2) A step of allowing the yeast in step (1) to express a yeast eRF3 having repetitions of the amino acid sequence as shown in SEQ ID NO: 2 in its prion domain

(3) A step of determining whether or not the test substance inhibits prionization of the yeast eRF3 expressed in step (2).

The “yeast eRF3 having repetitions of the amino acid sequence as shown in SEQ ID NO: 2 in its prion domain” used here may be either a yeast eRF3 that has repetitions of this sequence inherently, e.g., Y. lipolytica-derived eRF3 (YL eRF3), or a yeast eRF3 into which repetitions of this sequence have been integrated artificially, e.g., YLRP eRF3 or Δ121YLRP eRF3 described in Examples. Since YL eRF3 undergoes prionization at a high frequency (Table 3, Example 3), it is expected that other eRF3 proteins having repetitions of this sequence also undergo prionization at a high frequency. This nature of high susceptibility to prionization is a very effective nature in conducting screening for substances that inhibit transmission of abnormal PrP.

The determination of whether or not a test substance inhibits prionization of a yeast eRF3 can be performed by known methods. For example, since a prionized eRF3 form aggregates, these aggregates may be detected by a method using a fluorescent label, ultra-centrifugation, etc. Based on the results, whether the test substance inhibited prionization or not can be determined. When 74-D694Δsup35 strain is used as a yeast strain, the color of colonies formed by this yeast change depending on whether the eRF3 has been prionized or not. Thus, whether the test substance inhibited the conversion into the abnormal state can be determined with colony colors.

(D) Method of Detection of Abnormal PrP

As described above, it is highly possible that the transmission of eRF3 prionization via repetitions of the amino acid sequence as shown in SEQ ID NO: 2 is caused by a mechanism that is similar to the mechanism causing the transmission of abnormal PrP. Thus, it is believed that abnormal PrP in a sample can be detected by the following steps (1) and (2).

(1) A step of transferring a test sample into cells of a yeast comprising a yeast eRF3 in which repetitions of the amino acid sequence as shown in SEQ ID NO: 2 or a PrP sequence is integrated into its prion domain

(2) A step of determining whether the yeast eRF3 is prionized or not.

The “yeast eRF3 in which repetitions of the amino acid sequence as shown in SEQ ID NO: 2 are integrated into its prion domain” used here may be the same as described above in (C) Method of Screening for Substances that Inhibit Transmission of Abnormal PrP. Also, the determination of prionization of the yeast eRF3 may be performed in the same manner as described above in (C) Method of Screening for Substances that Inhibit Transmission of Abnormal PrP, e.g., determination of aggregate formation or colony colors.

The test sample used in this method is not particularly limited. For example, a part of bovine brain or spinal fluid, or beef may be enumerated.

A method of transferring the test sample into cells of the yeast is not particularly limited. For example, lipofection may be used.

(E) Method of Creation of Abnormal PrP

A yeast eRF3 in which repetitions of the amino acid sequence as shown in SEQ ID NO: 1 are integrated in its prion domain and a yeast eRF3 in which a part of the sequence in its prion domain is replaced with a polyglutamine sequence are able to prionize heterologous eRF3 proteins. Since yeast eRF3 and mammalian PrP share several characteristics in common, it is believed that normal PrP can be converted into abnormal PrP by contacting normal PrP with one of the above-described yeast eRF3 proteins.

Specific examples of the “yeast eRF3 in which repetitions of the amino acid sequence as shown in SEQ ID NO: 1 are integrated in its prion domain” include the eRF3 derived from K. lactis (KL eRF3) and chimeric eRF3 proteins ChiB and ChiC prepared in Example 5.

Specific examples of the “yeast eRF3 in which a part of the sequence in its prion domain is replaced with a polyglutamine sequence” include a peptide obtainable by replacing the oligonucleotide repeat region of Saccharomyces cerevisiae-derived eRF3 with polyglutamine.

The normal PrP used in this method is not particularly limited. Bovine or human PrP may be used, for example.

The amino acid sequence of the eRF3 of Saccharomyces cerevisiae is registered at the GenBank (Accession Number: M21129).

(F) Method of Screening for Substances that Restore Abnormal PrP to Normal PrP

It is believed that screening for substances that restore abnormal PrP to normal PrP can be performed by using the method of creation of abnormal PrP described in (E) above. The screening for those substances can be carried out by the following steps (1) to (3).

(1) A step of obtaining an abnormal PrP by contacting a normal PrP with a yeast eRF3 in which repetitions of the amino acid sequence as shown in SEQ ID NO: 1 are integrated into its prion domain or a yeast eRF3 in which a part of the sequence of its prion domain is replaced with a polyglutamine sequence,

(2) A step of contacting the abnormal PrP with a test substance

(3) A step of determining whether or not the test substance restores the abnormal PrP to a normal PrP.

The present specification encompasses the contents disclosed in the specification and/or drawings of Japanese Patent Application No. 2000-185039 based on which the present application claims priority.

### BRIEF DESCRIPTION OF THE DRAWINGS

FIG. 1 is a diagram showing homology between S. cerevisiae-derived eRF3 protein and eRF3 proteins derived from C. maltosa, D. hansenii, K. lactis, Y. lipolytica and Z. rouxii.

FIG. 2 is a diagram showing an outline of the colony assay using 74-D694Δsup35 strain.

FIG. 3 is a photograph showing the results of Example 3.

FIG. 4 presents photographs showing the results of Example 4.

FIG. 5 is a diagram showing the structures of chimeric eRF3 proteins prepared from S. cerevisiae-derived eRF3 and K. lactis-derived eRF3.

FIG. 6 presents photographs showing the results of Examples 6 and 7.

FIG. 7 presents photographs and a diagram showing the results of Example 8.

FIG. 8 presents photographs showing the results of Examples 9 and 10.

FIG. 9 is a diagram showing the structures of SC eRF3, YLRP eRF3 and Δ121YLRP eRF3.

FIG. 10 presents photographs showing the results of Example 11.

FIG. 11 is a diagram showing the structure of polyglutamine-replaced eRF3.

FIG. 12 presents photographs showing the results of Example 12.

FIG. 13 presents photographs showing the results of Example 13.

FIG. 14 presents photographs showing the results of Example 14.

FIG. 15 presents photographs showing the results of Example 15.

### BEST MODE FOR CARRYING OUT THE INVENTION

### EXAMPLE 1

### Isolation of Yeast eRF3 Genes

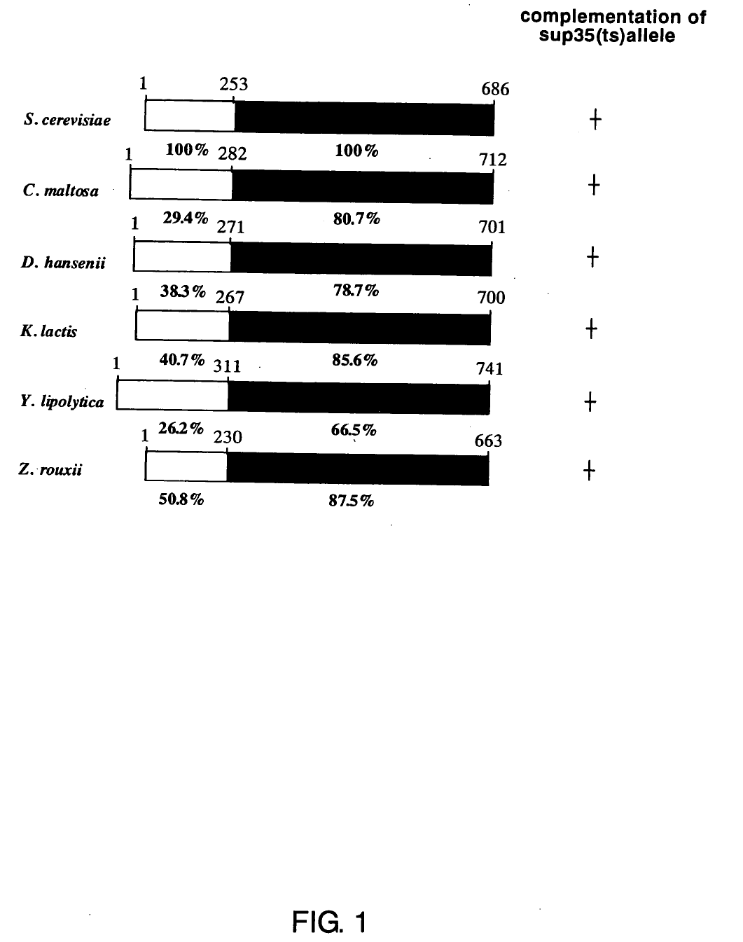

Five yeast species of Candida maltosa (IAM No.12247), Debaryomyces hansenii (IAM No. 12835), Kluyvermyces lactis (IAM No.12492), Yarrowia lipolytica (IAM No.4948) and Zygosaccharomyces rouxii (IAM No.12879) released from the Institute of Molecular and Cellular Biosciences, University of Tokyo, were grown to saturation in YPD medium. After harvesting, cells were suspended in 1 M sorbitol. Zymolyase-100T (Kirin Brewery) was added thereto at a final concentration of 0.5 mg/ml. The cells were treated at 37° C. for 1 hr, followed by addition of 1% SDS. The resultant sample was extracted with phenol-chloroform, subjected to ethanol precipitation, and then dissolved in TE. For each species, a genomic library was prepared from the resultant genomic DNA using λ DASHI I. Then, eRF3 gene was isolated by plaque hybridization. As a probe, a HpaI fragment (750-1236 nt) within S. cerevisiae eRF3 gene was used. DNA was prepared from the positive clones obtained, and the gene fragment was subcloned into pUC118, followed by determination of the nucleotide sequence. As a result, it was revealed that all of the resultant yeast eRF3 genes had the prion domain (N-terminal domain) required for causing prionization (FIG. 1).

Homology between these genes and S. cerevisiae-derived eRF3 gene was examined for the prion domain and the functional domain (C-terminal domain) (FIG. 1). As shown in FIG. 1, interspecific homology in the prion domain was very low, while homology in the functional domain was high.

### EXAMPLE 2

### Analysis of Yeast eRF3 Prion Domain

The oligopeptide repeats contained in the prion domain of individual yeast eRF3 proteins were searched. The search for oligopeptide repeats was performed by computer analysis of those sites having the same sequence against all subsequences consisting of 5-12 nucleotides contained in the amino acid sequences of the above-mentioned prion domain. For sequences consisting of 5 nucleotides, one mismatch was permitted; for sequences consisting of 6-9 nucleotides, up to two mismatches were permitted; and for sequences consisting of 10-12 nucleotides, up to 3 mismatches were permitted. The results are shown in Table 1. Given in parentheses are starting points of sequences having homology with relevant repeat motifs, though they were not scored in the computer analysis.

TABLE 1

| Species | Repeat Motif | Starting Point of Peptide Repeat | | | | | | |
|---|---|---|---|---|---|---|---|---|
| S. cerevisiae | YNPQGGYQQ | 55 | 63 | 73 | 82 | (92) | | |
| C. maltosa | GGYQQNYNNR | 63 | 75 | 85 | 95 | (105) | | |
| D. hansenii | GYQNYNQ | 71 | 79 | 86 | 96 | (104) | 119 | |
| | ATEKETTPA | 173 | 183 | 192 | 201 | 210 | 220 | 244 |
| K. lactis | QGYNAQQ | 34 | 44 | 61 | 75 | 82 | 117 | |
| Y. lipolytica | FVPGQS | 40 | 46 | 52 | | | | |
| | QGGYQGGYQGGY | 70 | 82 | 98 | 116 | 134 | | |
| | GGALKIGGDKP | 180 | 193 | 207 | 221 | 234 | | |
| | KESTP | 260 | 265 | 274 | | | | |
| Z. rouxii | GGYGGY | 36 | 42 | 48 | 54 | 72 | | |

As shown in Table 1, the presence of an inherent oligopeptide repeat(s) was confirmed in all of the species examined. In particular, Y. lipolytica-derived eRF3 was rich in repeat sequences throughout the N-terminal domain.

Subsequently, the ratios of Gln, Gly, Asn and Tyr in the amino acid residues contained in the N-terminal domain of each yeast eRF3 were determined. The results are shown in Table 2.

TABLE 2

| | Gln | Gly | Asn | Tyr |
|---|---|---|---|---|
| S. cerevisiae | 28.57 | 15.78 | 15.78 | 15.03 |
| C. maltosa | 35.41 | 11.11 | 12.50 | 12.50 |
| D. hansenii | 26.51 | 15.95 | 17.42 | 14.39 |
| K. lactis | 28.57 | 15.78 | 15.78 | 18.04 |
| Y. lipolytica | 22.72 | 29.22 | 7.79 | 13.63 |
| Z. rouxii | 27.18 | 24.27 | 8.73 | 15.53 |

As shown in Table 2, the ratios of these amino acids are high in the N-terminal domain. These four amino acid residues occupied more than 70% of the total amino acid residues.

### EXAMPLE 3

### Detection of the Prion-Like Properties of Yeast eRF3

eRF3 is a subunit of a polypeptiderelease factor. If eRF3 has been prionized, this protein becomes unable to recognize a normal stop codon. As a result, it possesses a weak, nonsense suppressor activity ([PSI⁺]). S. cerevisiae 74-D694 strain (Chernoff et al., 1995) is a strain in which this suppressor activity can be detected. This strain has a nonsense mutation (adel-14(UGA)) in its adel gene involved in adenine synthesis. Under usual conditions, adenine precursor is accumulated in cells of this strain, and red colonies are formed. However, when eRF3 has been prionized, adel gene product begins to be synthesized due to the suppressor activity. As a result, white colonies are formed. Thus, it is possible to judge whether the eRF3 in this strain is prionized (i.e., converted into [PSI⁺]) or not with the color of colonies formed by this strain.

In order to examine whether yeast eRF3 proteins derived from species other than S. cerevisiae are also converted into [PSI⁺] or not, the following experiment was conducted using the above-described S. cerevisiae 74-D694 strain.

First, the chromosomal eRF3 gene of S. cerevisiae 74-D694 strain was deleted to thereby prepare a strain (74-D694Δsup35) in which its own eRF3 is not expressed. This 74-D694Δsup35 strain was prepared as described below. Briefly, a PvuII-XbaI 3.4 kb DNA fragment containing S. cerevisiae eRF3 gene was cloned into the HincII-XbaI site of pHSG399 (to prepare pHSGsup35), followed by replacement of the EcoRV-SalI fragment contained in this gene fragment with LEU2 marker (pHSGΔsup35::LEU2). The gene fragment cloned into this plasmid was amplified by PCR using a universal primer set, and then transformed into 74-D694 strain retaining the wild-type eRF3 gene in a plasmid (pRS316). LEU⁺ clones were selected, and it was confirmed by genomic PCR that the eRF3 gene had been disrupted.

Subsequently, plasmids for expressing individual yeast eRF3 genes were introduced into the resultant 74-D694Δsup35 strain, which was then cultured on YPD medium to examine the color of its colonies. The plasmids for expressing individual yeast eRF3 genes were prepared by amplifying individual eRF3 genes by PCR using the following primers and universal primers, and then cloning the amplified fragments into the BamHI site or BamHI-EcoRI site downstream of TPI promoter of pYX112 or pRS313.

| | | |
|---|---|---|
| C. maltosa: | 5′-CCGGATCCATATGTCTAACCCTCAAGATCA-3′ | (SEQ ID NO: 3) |
| D. hansenii: | 5′-CCGGATCCATATGTCTGACGATCAACAGTA-3′ | (SEQ ID NO: 4) |
| K. lactis: | 5′-CCGGATCCATATGTCAGACCAACAAAATCA-3′ | (SEQ ID NO: 5) |
| Y. lipolytica: | 5′-CCGGATCCATATGAGTGATCAATTCAACCA-3′ | (SEQ ID NO: 6) |
| Z. rouxii: | 5′-CCGGATCCATATGTCTGACCCAAACCAGAA-3′ | (SEQ ID NO: 7) |

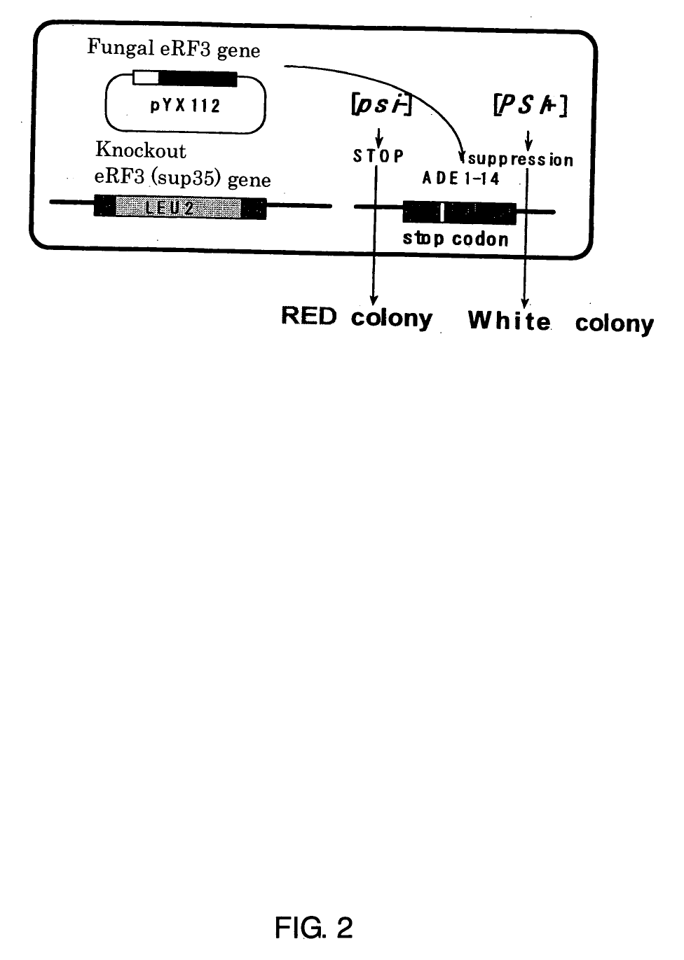

The thus manipulated 74-D694Δsup35 strain forms white colonies when the exogenous eRF3 is converted into [PSI⁺], and forms red colonies when the exogenous eRF3 is not converted into [PSI⁺] (also expressed as [psi]) (FIG. 2). The frequency of appearance of white colonies when the exogenous eRF3 gene has been expressed in 74-D694Δsup35 strain is shown in Table 3 (spontaneous). It is known that excessive expression of the prion domain of eRF3 induces conversion into [PSI⁺] (Ter-Avanesya et al., 1993; Chemoff et al., 1993; Derkatch et al., 1996). Then, while expressing the exogenous eRF3 gene in 74-D694Δsup35 strain, the prion domain of eRF3 of the same species to the host was allowed to be expressed excessively using pYES. Under these conditions, the frequency of appearance of white colonies was also examined (Table 3; induced).

TABLE 3

| | SC | CM | DH | KL | YL | ZR |
|---|---|---|---|---|---|---|
| spontaneous | 10⁻⁷ | 10⁻⁶ | 10⁻⁷ | 10⁻⁵ | 10⁻¹ | 10⁻⁶ |
| induced | 10⁻⁴ | 10⁻⁵ | 10⁻⁶ | 10⁻³ | 10⁻¹ | 10⁻⁵ |

SC: S. cerevisiae,
CM: C. maltosa,
DH: D. hansenii,
KL: K. lactis,
YL: Y. lipolytica,
ZR: Z. rouxii

As shown in Table 3, conversion into [PSI⁺] occurred at a very high frequency (10⁻¹) when the eRF3 of Y. lipolytica was expressed, regardless of the presence or absence of the excessive expression of the prion domain.

It is known that yeast eRF3 proteins converted into [PSI⁺] are restored to normal eRF3 by addition of guanidine hydrochloride. Then, 3 mM guanidine hydrochloride was added to a plate to examine changes in colony color.

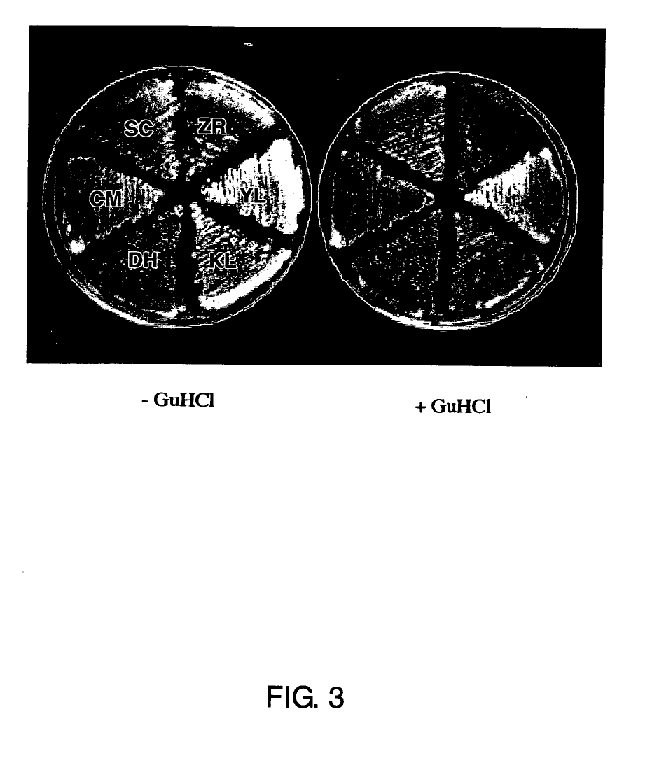

The photograph at the left side of FIG. 3 shows a plate on which exogenous eRF3 proteins were expressed, followed by selection of ADE⁺ colonies. The photograph at the right side of FIG. 3 shows the same plate after addition of guanidine hydrochloride. As shown in these photographs, colony color turned red in all the strains except the one into which Y. lipolytica eRF3 gene had been transferred. From these results, it was confirmed that the colony color turned white was caused by the conversion of the introduced eRF3 into [PSI⁺]. The strain into which Y. lipolytica eRF3 gene had been transferred formed pink colonies, not red colonies.

### EXAMPLE 4

### Assay of Cross-Species Transmissibility in Yeast Prion

In PrP, the presence of a species barrier for cross-species transmission is well-known. In eRF3 proteins converted into [PSI⁺], a species barrier for cross-species transmission has also been confirmed between S. cerevisiae and P. methanolica (Kushnirov et al., 2000; Chernoff et al., 2000) and between S. cerevisiae and C. albicans or K. lactis (Santoso et al., 2000).

In order to further investigate into a species barrier for cross-pecies transmission of [PSI⁺] conversion in eRF3 proteins, the experiment described below was conducted.

Briefly, eRF3 proteins from S. cerevisiae , C. maltosa, D. hansenii, K. lactis, Y. lipolytica and Z. rouxii were expressed separately using pYX112 in S. cerevisiae 74-D694Δsup35 clones obtained in Example 3 whose phenotypes were rendered [PSI⁺] by expressing eRF3 proteins from K. lactis, Y. lipolytica and D. hansenii, respectively. Subsequently, the clones were cultured on YPD medium to examine the color of colonies formed. When the newly expressed eRF3 is not converted into [PSI⁺] because of a species barrier, red colonies are formed. On the other hand, when the newly expressed eRF3 is also converted into [PSI⁺], white colonies are formed.

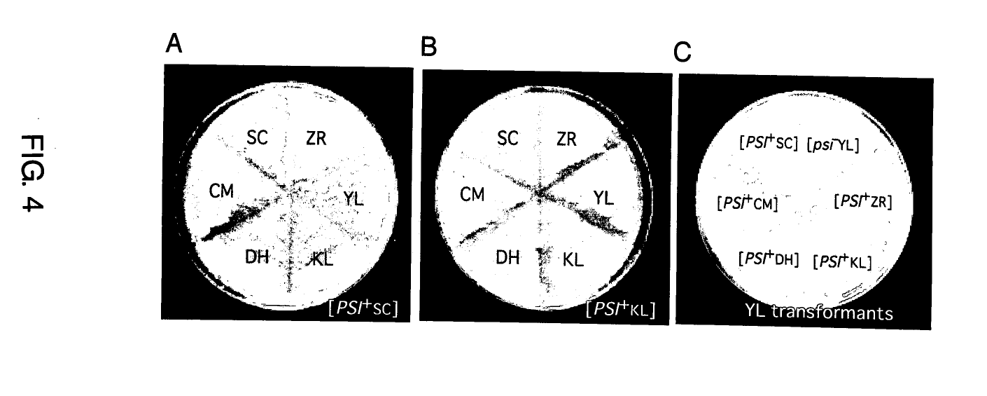

FIG. 4A shows the states of colonies when individual yeast eRF3 proteins were introduced into a clone expressing [PSI⁺] eRF3 of S. cerevisiae. FIG. 4B shows the states of colonies when individual yeast eRF3 proteins were introduced into a clone expressing [PSI⁺] eRF3 of K. lactis. FIG. 4C shows the states of colonies when eRF3 of Y. lipolytica was introduced into clones expressing individual yeast [PSI⁺] eRF3 proteins. The number of colonies (per 100 colonies) that are white and become red by the addition of guanidine hydrochloride is shown in Table 4.

TABLE 4

| | | [PSI⁺] conversion-induced eRF3 | | | | | |
|---|---|---|---|---|---|---|---|
| | | S. cerevisiae | C. maltosa | D. hansenii | K. lactis | Y. lipolytica | Z. rouxii |
| [PSI⁺] conversion- | S. cerevisiae | 100 | 0 | 0 | 0 | 100 | 0 |
| inducing eRF3 | K. lactis | 67 | 58 | 70 | 95 | 100 | 68 |
| | Y. lipolytica | 0 | 0 | 0 | 0 | 100 | 0 |
| | D. hansenii | 0 | 0 | 83 | 0 | 87 | 0 |
| | control | 0 | 0 | 0 | 0 | 10 | 0 |

As shown in Table 4, a species barrier for transmission exists between two species in many cases. However, special natures were observed in K. lactis eRF3 and Y. lipolytica eRF3. K. lactis eRF3 induced [PSI⁺] conversion in all the eRF3 proteins of the species used in this experiment at high rates (FIG. 4B). On the other hand, in Y. lipolytica eRF3, [PSI⁺] conversion was induced at high rates by all the eRF3 proteins of the species used in this experiment (FIG. 4C).

### EXAMPLE 5

### Mechanism of Cross-Species Transmission in K. lactis eRF3

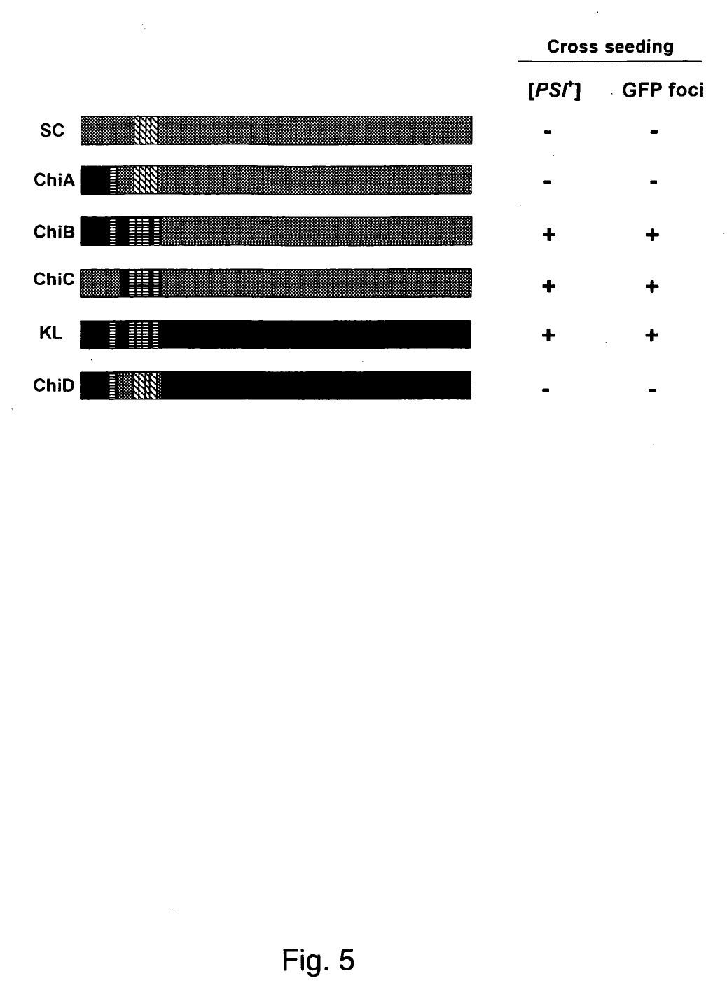

No characteristic subsequences are found in the prion domain of K. lactis eRF3. In order to identify a cis-element involved in the strong transmissibility exhibited by [PSI⁺] K. lactis eRF3, the following four types of chimeric eRF3 proteins from K. lactis eRF3 and S. cerevisiae eRF3 were constructed (FIG. 5).

1) ChiA

The gene of this chimeric protein was prepared by replacing the BamHI-PstI fragment of S. cerevisiae sup35 gene with a K. lactis sup35 gene fragment amplified with the following primers.

| | |
|---|---|
| 5′-GGGGATCCAATGTCAGACCAACAAAATCA-3′ | (SEQ ID NO: 8) |
| 5′-GGGCTGCAGGAGCACCTTGTTGGCCGTTGT-3′ | (SEQ ID NO: 9) |

2) ChiB

The gene of this chimeric protein was prepared by replacing the BamHI-EcoRV fragment of S. cerevisiae sup35 gene with a K. lactis sup35 gene fragment amplified with the following primers.

| 5′-GGGGATCCAATGTCAGACCAACAAAATCA-3′ | (SEQ ID NO: |
|---|---|
| | 10) |
| 5′-GGGGATATCCGGTTGGCTGTTGTGCATTAT-3′ | (SEQ ID NO: |
| | 11) |

3) ChiC

The gene of this chimeric protein was prepared by replacing the PstI-EcoRV fragment of S. cerevisiae sup35 gene with a K. lactis sup35 gene fragment amplified with the following primers.

| 5′-GGGCTGCAGGCTACCAAGCATATCAAGCTT-3′ | (SEQ ID NO: |
|---|---|
| | 12) |
| 5′-GGGCTGCAGGAGCACCTTGTTGGCCGTTGT-3′ | (SEQ ID NO: |
| | 13) |

4) ChiD

The gene of this chimeric protein was prepared by replacing the EcoRV-XhoI fragment of ChiA gene with a K. lactis sup35 gene fragment amplified with the following primers.

| 5′-GGGATATCAAGCTCCAGCACAGTCTTCATC-3′ | (SEQ ID NO: |
|---|---|
| | 14) |
| 5′-GGGCTCGAGTTAATTTTCAAGGATTTTCAC-3′ | (SEQ ID NO: |
| | 15) |

Whether the thus constructed four chimeric eRF3 proteins convert heterologous eRF3 proteins into [PSI⁺] or not was judged by two methods based on colony color and formation of aggregates.

Judgment based on colony color was conducted as follows. Briefly, each chimeric eRF3 gene was introduced into 74-D694Δsup35 strain using pRS313 to render the phenotype of this strain [PSI⁺]. D. hansenii eRF3 gene was introduced into the resultant clones using pYX112, followed by cultivation on YPD medium and colony formation. When white colonies were formed and they turned red by the addition of guanidine hydrochloride, the relevant chimeric eRF3 was judged “transmissible”.

Judgment based on the formation of aggregates was conducted as follows. Briefly, each chimeric eRF3 gene was introduced into 74-D694Δsup35 strain to render the phenotype of this strain [PSI⁺]. To the resultant clones, a sequence for expressing a fusion protein composed of D. hansenii eRF3 and GFP was introduced using pYX112. Then, the states of resultant cells were observed with a fluorescent microscope. Since eRF3 converted into [PSI⁺] forms aggregates, the relevant chimeric eRF3 was judged “transmissible” when fluorescent spots (aggregates) were observed with the microscope.

As control experiments, the same experiments were conducted using K. lactis eRF3 and S. cerevisiae eRF3 instead of chimeric eRF3 proteins. The results are shown in Table 5.

TABLE 5

| | | Judgment based |
|---|---|---|
| | | on aggregate |
| eRF3 | Judgment based on colony color | formation |
| S. cerevisiae | non-transmissible | non-transmissible |
| ChiA | non-transmissible | non-transmissible |
| ChiB | transmissible | transmissible |
| ChiC | transmissible | transmissible |
| K. lactis | transmissible | transmissible |
| ChiD | non-transmissible | non-transmissible |

As shown in Table 5, two chimeric eRF3 proteins (ChiB and ChiC) induced [PSI⁺] conversion of heterologous yeast eRF3. Both of these chimeric eRF3 proteins contain a fragment (43-126 nt) containing the oligopeptide repeat of K. lactis eRF3. On the other hand, neither ChiA nor ChiD, which does not contain this fragment, exhibits transmissibility. Therefore, it is presumed that the fragment containing the oligopeptide repeat of K. lactis eRF3 is essential for converting heterologous yeast eRF3 into [PSI⁺].

### EXAMPLE 6

### Assay of Transmissibility of [PSI⁺] K. lactis eRF3

The experiment described below was conducted to confirm that [PSI⁺] K. lactis eRF3 converts other eRF3 proteins into [PSI⁺].

First, K. lactis eRF3 gene was introduced into 74-D694Δsup35 strain using pRS313 to render the phenotype of this strain [PSI⁺]. Subsequently, genes that express fusion proteins composed of the prion domain of S. cerevisiae, K. lactis or D. hansenii eRF3 and GFP were prepared as described below.

1) Gene that Expresses a Fusion Protein Composed of the Prion Domain of S. cerevisiae eRF3 and GFP (Designated NMSC-GFP)

This gene was prepared by replacing the HpaI-SacI fragment of pYX112sup35SC with a GFP gene fragment amplified using the following primers and pEGFP (Clontech) as a template.

| 5′-GGGGTCGACATGGTGAGCAAGGGCGAGGAG-3′ | (SEQ ID NO: |
|---|---|
| | 16) |
| 5′-GGGGAGCTCTTACTTGTACAGCTCGTCCA-3′ | (SEQ ID NO: |
| | 17) |

2) Gene that Expresses a Fusion Protein Composed of the Prion Domain of K. lactis eRF3 and GFr (Designated NMKL-GFP)

This gene was prepared by cloning the K. lactis eRF3 prion domain amplified with the following primers into the BamHI-SalI site of pYX112, and then cloning the above-described GFP gene fragment into the SalI-SacI site of this plasmid.

| 5′-CCGGATCCATATGTCAGACCAACAAAATCA-3′ | (SEQ ID NO: |
|---|---|
| | 18) |
| 5′-GGGGGTCGACATCTTTAACGACTTCTTCGT-3′ | (SEQ ID NO: |
| | 19) |

3) Gene that Expresses a Fusion Protein Composed of the Fusion Domain of D. hansenii eRF3 and GFP (Designated NMDH-GFP)

This gene was prepared by cloning the D. hansenii eRF3 prion domain amplified with the following primers into the BamHI-SalI site of pYX112, and then cloning the above-described GFP gene fragment into the SaIlI-SacI site of this plasmid.

| 5′-CCGGATCCATATGTCTGACGATCAACAGTA-3′ | (SEQ ID NO: |
|---|---|
| | 20) |
| 5′-GGGGGTCGACATCCTTGACAACTTCTTCAT-3′ | (SEQ ID NO: |
| | 21) |

The thus prepared genes were introduced separately into the [PSI⁺] 74-D694Δsup35 strain described above, followed by observation with a fluorescent microscope.

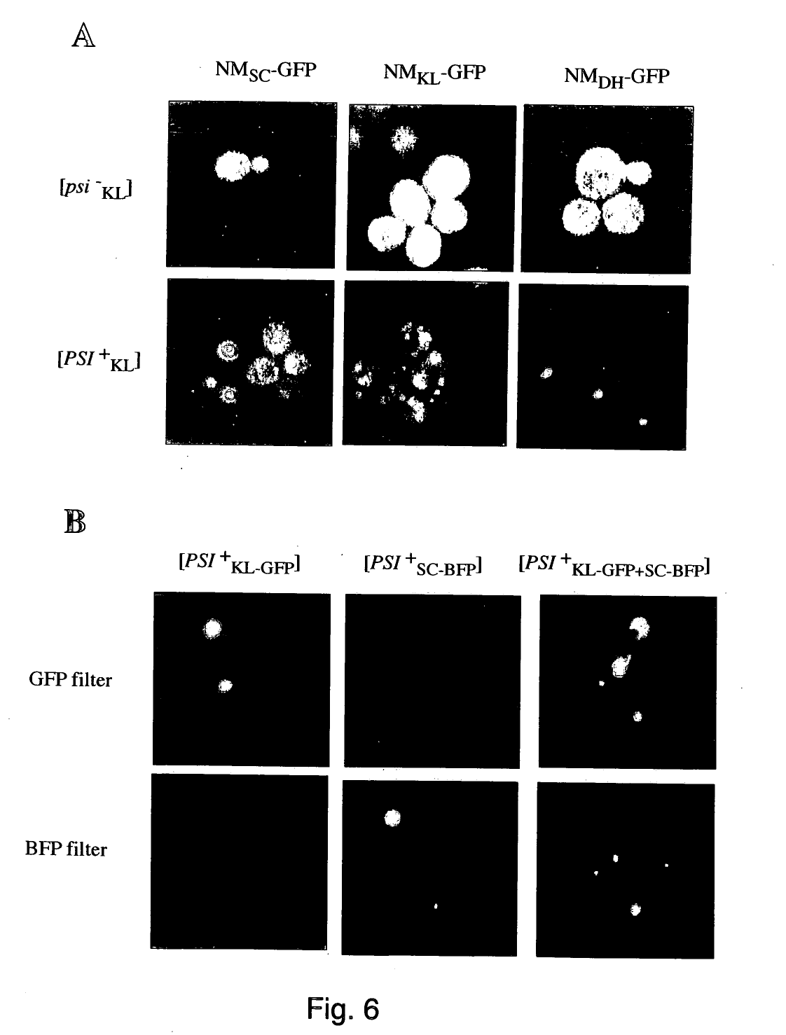

As a control experiment, 74-D694Δsup35 strain having [psi] eRF3 was manipulated in the same manner. The results are shown in FIG. 6A.

In FIG. 6A, the upper panels are photographs showing the results when [psi] 74-D694Δsup35 strain was used, and the lower panels are photographs showing the results when [PSI⁺] 74-D694Δsup35 strain was used. As these photographs show, in 74-D694Δsup35 strain having [PSI⁺] eRF3, expressed fusion proteins formed aggregates. Thus, the [PSI⁺] phenotype of the strain was maintained.

### EXAMPLE 7

### Confirmation of Contact between eRF3 Proteins

Whether a [PSI⁺] eRF3 is in contact with a newly introduced eRF3 or not was determined with GFP/BFP double fluorescence.

First, a gene that expresses a fusion protein composed of K. lactis eRF3 and GFP (KL-GFP) and a gene that expresses a fusion protein composed of S. cerevisiae eRF3 and BFP (SC-BFP) were prepared as described below.

SC-BFP: A gene for this protein was prepared by replacing the SalI-SacI fragment of pYX112Sup35SC with a BFP gene fragment amplified using the following primers and pQBI50 (Takara) as a template.

| 5′-GGGGTCGACATGGCTAGCAAAGGAGAAGAA-3′ | (SEQ ID NO: |
|---|---|
| | 22) |
| 5′-GGGGAGCTCGATCCTTATTTGTAT-3′ | (SEQ ID NO: |
| | 23) |

KL-GFP: A gene for this protein was prepared by replacing the BamHI-StuI gene fragment of pRS313TPIsup35SC with a K. lactis sup35 gene fragment amplified using the following primers, and then replacing the Sal-SacI gene fragment of the this plasmid with the above-described GFP gene fragment.

| 5′-GGGGATCCAATGTCAGACCAACAAAATCA-3′ | (SEQ ID NO: |
|---|---|
| | 24) |
| 5′-GGGAGGCCTTACCCACTTCAATGGTTTTAC-3′ | (SEQ ID NO: |
| | 25) |

Subsequently, the gene that expresses KL-GFP was introduced into 74-D694Δsup35 strain using pRS313 to render the phenotype of this strain [PSI⁺]. Then, the gene that expresses SC-BFP was introduced into the resultant strain using pYX112. The thus manipulated 74-D694Δsup35 strain was observed with a fluorescent microscope (Olympus BH2-RFC) and photographed with a CCD camera (Olympus C-3030). The results are shown in the two photographs provided at the right side of FIG. 6B. The upper photograph was taken with DMIB filter (Olympus: excitation 490 nm, emission 515 nm) so that only the fluorescence from GFP can be observed. The lower photograph was taken with XE113-2 filter (Omega Optical: excitation 387 nm, emission 450 nm) so that only the fluorescence from BFP can be observed. As these photographs show, the locations of aggregates labeled with GFP completely coincided with the locations of aggregates labeled with BFP. These results revealed that [PSI⁺] K. lactis eRF3 is in direct contact with S. cerevisiae eRF3.

The two panels at the left side of FIG. 6B are fluorescent microphotographs showing 74-D694Δsup35 strain converted into [PSI⁺] phenotype by introduction of the gene that expresses KL-GFP. The two panels at the center of FIG. 6B are fluorescent microphotographs showing 74-D694Δsup35 strain converted into [PSI⁺] phenotype by introduction of the gene that expresses SC-BFP.

### EXAMPLE 8

### Assay of Phenotypes after Removal of the Source of Transmission

S. cerevisiae eRF3 gene was introduced into 74-D694Δsup35 strain using pRS313 to render the phenotype of this strain [PSI⁺] (step 1). To this strain, Y. lipolytica eRF3 gene was introduced using pYX112 (step 2). After a certain period of cultivation, those clones were selected from which the plasmid comprising S. cerevisiae eRF3 gene had been dropped off (step 3).

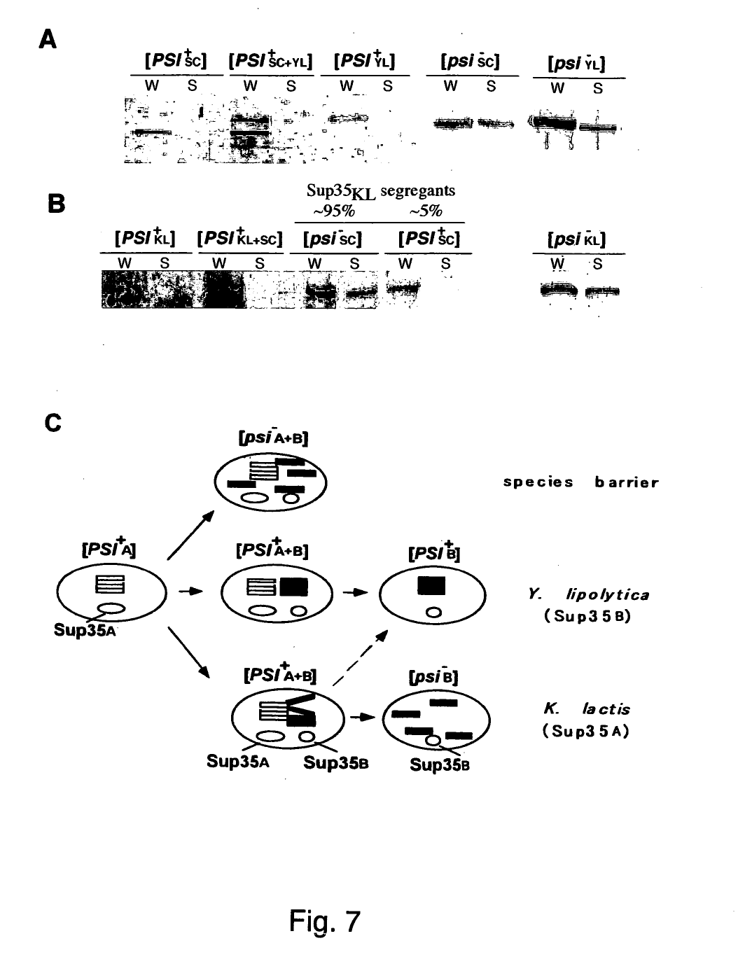

The lysate of the 74-D694Δsup35 strain from each step was separated into whole lysate (W) and supernant (S) by ultra-centrifugation (100,000 g). The eRF3 proteins contained in both fractions were detected with antibodies. The results are shown in FIG. 7A. The expressions [PSI⁺SC], [PSI⁺SC+YL] and [PSI⁺YL] appearing in this Figure correspond to step 1, step 2 and step 3, respectively. As shown in these photographs, eRF3 is only detected in whole lysate fraction in any of the steps 1 to 3, and not detected in supernant fraction. Since [PSI⁺] eRF3 forms huge aggregates, it is not detected in supernant fraction. Therefore, these results means that the phenotype of the 74-D694Δsup35 strain was [PSI⁺] in any of the steps (1) to (3).

K. lactis eRF3 gene was introduced into 74-D694Δsup35 strain using pRS313 to render the phenotype of this strain [PSI⁺] (step 1). To this strain, S. cerevisiae eRF3 gene was introduced using pYX112 (step 2). After a certain period of cultivation, those clones were selected from which the plasmid comprising K. lactis eRF3 gene had been dropped off (step 3).

The lysate of the 74-D694Δsup35 strain from each step was separated into whole lysate (W) and supernant (S) in the same manner as described above, followed by detection of the eRF3 proteins contained in both fractions. The results are shown in FIG. 7B. The expressions [PSI⁺KL] and [PSI⁺KL+SC] appearing in this Figure correspond to step 1 and step 2, respectively, and the expression “Sup35KL segregants” responds to step 3. As shown in these photographs, eRF3 is only detected in whole lysate fraction in steps (1) and (2), indicating that the 74-D694Δsup35 strain retains the phenotype [PSI⁺]. In step 3, however, eRF3 is also detected in supernant fraction in about 95% of the clones, indicating that the phenotype has been changed to [psi].

As described above, when Y. lipolytica eRF3 is converted into [PSI⁺] using S. cerevisiae eRF3, the [PSI⁺] phenotype is retained even after removal of the source of transmission (i.e., S. cerevisiae eRF3). On the contrary, when S. cerevisiae eRF3 is converted into [PSI⁺] using K. lactis eRF3, the phenotype changes to [psi] after removal of the source of transmission (i.e., K. lactis eRF3).

These results suggest that, when S. cerevisiae eRF3 and K. lactis eRF3 co-exist in cells, aggregates of K. lactis eRF3 form cores and S. cerevisiae eRF3 is in contact with such aggregates (FIG. 7C). When the cores are removed, S. cerevisiae eRF3 is presumed to be released from aggregation, changing the phenotype of the host clone from [PSI⁺] to [psi]. In several percent of the clones used, [PSI⁺] phenotype is retained stably even after removal of K. lactis eRF3. This means that K. lactis eRF3 converted into [PSI⁺] has an ability to cause cross-species transmission.

### EXAMPLE 9

### Analysis of [PSI⁺] K. lactis eRF3

Since K. lactis eRF3 converted into [PSI⁺] exhibited rather different natures from other [PSI⁺] eRF3 proteins, a detailed analysis was performed.

It is known that S. cerevisiae eRF3 converted into [PSI⁺] requires the presence of HSP104 for retaining this character (Chernoff et al., 1995). In order to examine whether K. lactis eRF3 also shows the same nature or not, the following experiment was conducted.

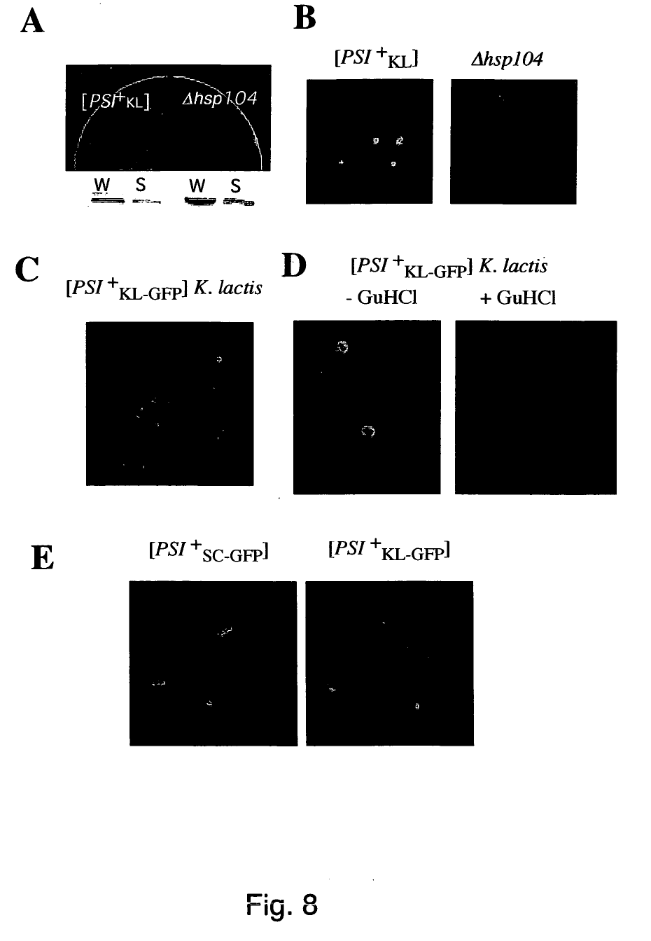

In [PSI⁺] 74-D694Δsup35 strain into which K. lactis eRF3 gene was introduced using pRS314, hsp104 gene was disrupted. Briefly, hsp104-disrupted 74-D694Δsup35 strain was prepared by introducing into 74-D694Δsup35 strain a DNA fragment (Δhsp104::HIS3) obtained by replacing the BglII-EcoRV fragment in the 3.3 kb DNA fragment containing hsp104 with HIS3 marker, selecting HIS⁺ transformants, and confirming the disruption by genomic PCR. The resultant 74-D694Δsup35 strain was cultured on YPD medium, followed by examination of colony color. Also, the lysate of the 74-D694Δsup35 strain was separate into whole lysate (W) and supernant (S), followed by detection of eRF3 proteins with antibodies. The results are shown in FIG. 8A. The two lanes at the right side of FIG. 8A represent hsp104-disrupted colonies, and the two lanes at the left side of FIG. 8A represent non-hsp104-disrupted colonies. As shown in this Figure, when hsp104 is disrupted, red colonies are formed, and eRF3 is also detected in supernant (S) fraction. That is, like S. cerevisiae eRF3, K. lactis eRF3 also could not retain the [PSI⁺] phenotype in the absence of HSP104.

Further, after the disruption of hsp104 in 74-D694Δsup35 strain, a gene that expresses a fusion protein composed of K. lactis eRF3 and GFP was introduced into this strain, followed by observation of the state of cells with a fluorescent microscope. The results are shown in FIG. 8B. The right panel of FIG. 8B represents hsp104-disrupted cells and the left panel non-hsp104-disrupted cells. As shown in these panels, aggregates were not formed in hsp104-disrupted cells. These results also indicate that HSP104 is essential for retaining the [PSI⁺] phenotype.

### EXAMPLE 10

### Assay of [PSI⁺] Conversion in K. lactis Cells

In the experiments so far described, all the [PSI⁺] conversion of eRF3 took place in S. cerevisiae cells. In order to examine whether the [PSI⁺] conversion of eRF3 occurs in K. lactis cells in the same manner or not, the following experiment was conducted.

A gene that expresses a fusion protein composed of K. lactis eRF3 and GFP was introduced into K. lactis SAY119 strain (MATa uraA1, trp1, leu2, ade1, metA1; Astrom et al., 2000) and expressed in excess. The thus manipulated K. lactis SAY119 strain was observed with a fluorescent microscope. As a result, formation of aggregates as shown in FIG. 8C was confirmed, though at a low frequency (about 1/1000). This strongly suggests that K. lactis eRF3 can be converted into [PSI⁺] even in K. lactis cells.

In K. lactis, no strains such as 74-D694Δsup35 suitable for detection of [PSI⁺] phenotype are known. Then, the inventors introduced the adel-14 gene of 74-D694Δsup35 strain into K. lactis SAY119 strain, to thereby created a strain in which [PSI⁺] conversion can be detected by colony color. To the thus created strain, a gene that expresses a fusion protein composed of K. lactis eRF3 and GFP was introduced and expressed in excess. As a result, white colony-forming clones appeared at a frequency of 10⁻⁵. In ⅜ of these clones, formation of aggregates was confirmed (FIG. 8D; left panel). When these aggregate-forming clones were cultured in a medium containing guanidine hydrochloride, the aggregates disappeared (FIG. 8D; right panel). This fact also suggests that K. lactis eRF3 can be converted into [PSI⁺] in K. lactis cells.

Furthermore, the inventors examined whether the nature of [PSI⁺] K. lactis observed this time (i.e., forming aggregates) can only be observed by using TPI promoter (a high expression promoter); or whether that nature can also be observed with a low expression promoter.

A gene that expresses a fusion protein composed of K. lactis eRF3 and GFP was introduced into 74-D694Δsup35 strain using a low expression vector pYX11 (with 786 promoter: Novagen), followed by observation of the state of cells with a fluorescent microscope. As a result, formation of aggregates (though small in size) was confirmed (FIG. 8E, right panel). The small size of these aggregates is presumed to be attributable to low expression level of K. lactis eRF3 that would form cores for aggregates. These results indicated that the nature of [PSI⁺] K. lactis eRF3 of forming aggregates does not depend on the expression level alone, and that this nature is attributable to the amino acid sequence of K. lactis eRF3.

### EXAMPLE 11

### Analysis of Repeat Sequences in Y. lipolytica

The N-terminal domain of Y. lipolytica eRF3 is rich in repeat sequences. Among all, repetitions of the repeat unit GGALKIGGDKP starting from the 180th residue are remarkable in length and perfect repeat of the unit. It is also interesting that this repeat sequence does not contain any Q/N. Q/N-rich sequence is a feature found in yeast prion but not found in mammalian PrP. Analysis of this repeat sequence containing no Q/N is interesting because the results will emphasize common features between Y. lipolytica eRF3 and PrP.

Whether this repeat sequence containing no Q/N (referred to as YLRP) facilitates [PSI⁺] conversion or not was examined using S. cerevisiae eRF3.

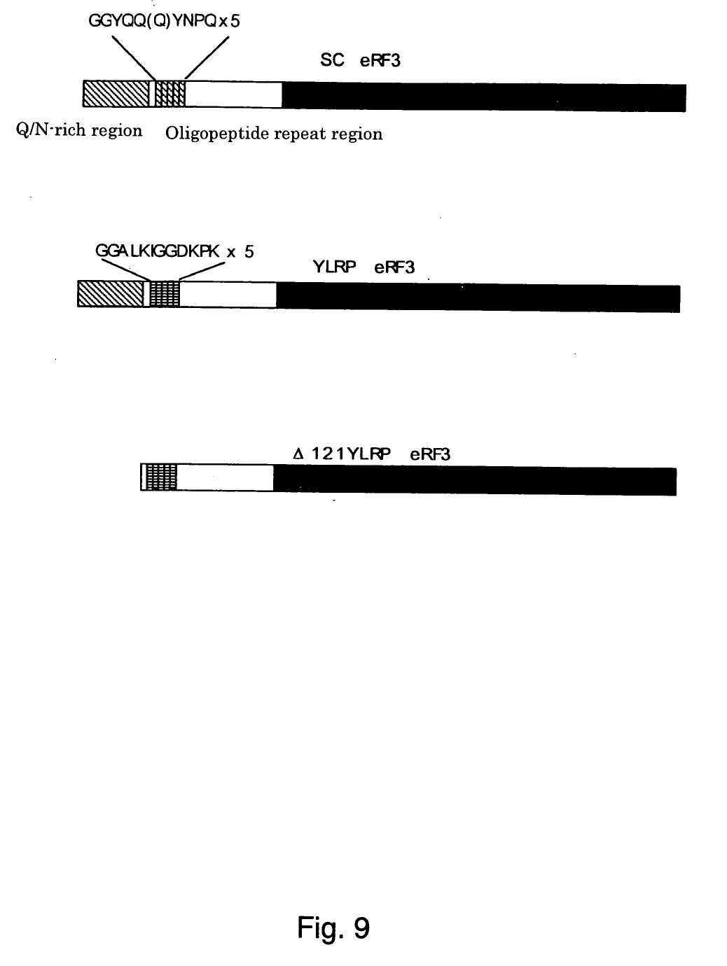

First, the oligopeptide repeat region of S. cerevisiae eRF3 (containing Q/N) was replaced with this repeat sequence containing no Q/N to thereby prepare YLRP eRF3 (FIG. 9B). This replacement was performed by replacing the PstI-EcoRV fragment of S. cerevisiae eRF3 gene with a fragment of Y. lipolytica eRF3 gene amplified using the following primers.

| 5′-GGGCTGCAGCTCTCAACAAGCTCAAGAAGC-3′ | (SEQ ID NO: |
|---|---|
| | 26) |
| 5′-GGGGATATCCCTCCTTCTTCTCGCTCTCCT-3′ | (SEQ ID NO: |
| | 27) |

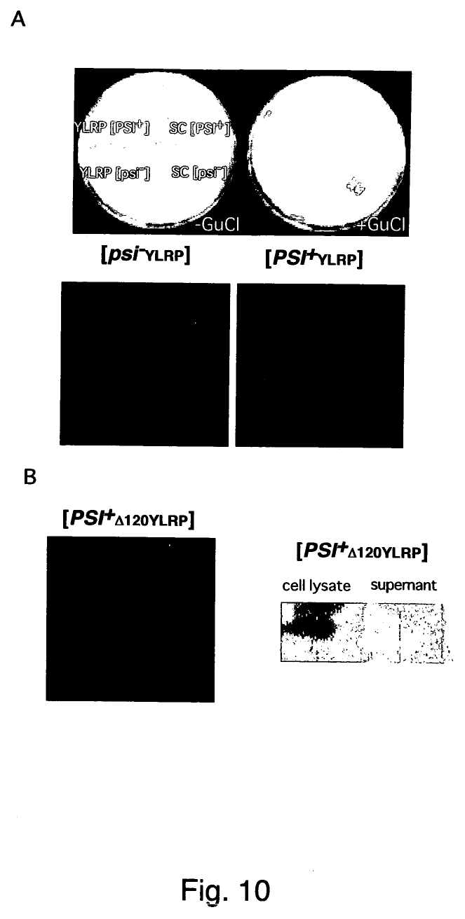

Subsequently, the gene that expresses the above-described YLRP eRF3 was introduced into 74-D694Δsup35 pYX112sup35 [PSI⁺] ([PSI⁺SC]) strain, followed by selection of ADE⁺ clones. The color of resultant colonies was observed, and the state of YLRP-BFP fusion protein was examined with a fluorescent microscope. The results are shown in FIG. 10A. As shown in this Figure, white colonies were formed by the introduction of YLRP eRF3. The formation of aggregates was also confirmed in the cells.

As Liu and Lindquist have already reported, a Q/N-containing repeat region is essential for retaining a stable [PSI⁺] phenotype. However, it has become clear that YLRP eRF3 is able to retain [PSI⁺] phenotype, though it does not contain such a Q/N-rich repeat sequence.

S. cerevisiae repeat region contains a Q-rich region at the N-terminal. A modified protein Δ120YLRP eRF3 was created by deleting this region (FIG. 9C). Surprisingly, this modified protein was converted into [PSI⁺] at a very high probability. The [PSI+] phenotype of this protein was confirmed by colony color, a fusion protein with BFP, and ultra-centrifugation assay (FIG. 10B). This phenomenon suggests a common feature with PrP in a sense that [PSI+] phenotype is retained by a repeat structure alone that does not contain Q/N-rich sequence.

### EXAMPLE 12

### Assay of Transmissibility of Polyglutamine-Replaced eRF3

A DNA fragment was amplified using a synthetic DNA 5′-GGGCTGCAGG CCAGCAACAA CAGCAGCAGC AGCAACAACA GCAACAACAG CAACAGCAAC AACAACAGCA ACAGCAACAG CAGCAGCAAC AGCAACAACA GCAACAGCAA CAGCAGCAAC AACAACAATA CGGATATCCC CC-3′ (SEQ ID NO: 28) as a template and the following primers.

| | |
|---|---|
| 5′-GGGCTGCAGGCCAGC-3′ | (SEQ ID NO: 29) |
| 5′-GGGGGATATCCGTAT-3′ | (SEQ ID NO: 30) |

The resultant DNA fragment was cloned into the PstI-EcoRV site of the wild-type eRF3 gene of S. cerevisiae.

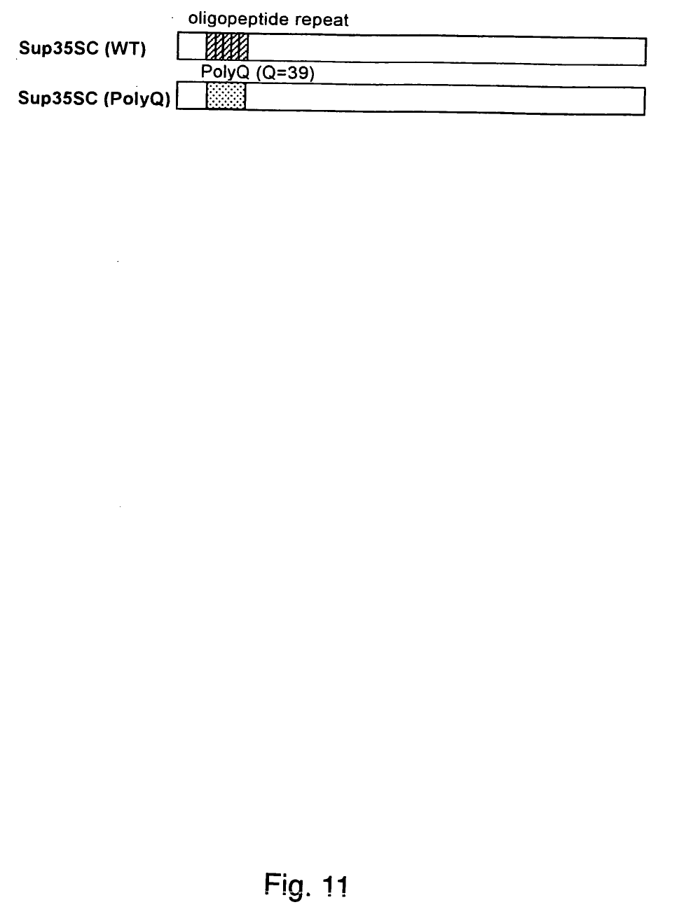

The thus modified eRF3 gene codes for an eRF3 protein in which the oligopeptide repeat region (44-111) of S. cerevisiae wild-type eRF3 is replaced with a polyglutamine sequence (Poly Q; Q=39) (referred to as polyglutamine-eplaced eRF3) (FIG. 11).

This gene for polyglutamine-eplaced eRF3 was introduced into 74-D694Δsup35 strain to render the phenotype of this strain [PSI⁺]. To this yeast strain, genes encoding fusion proteins each composed of the prion domain of K. lactis, D. hansenii or Z. rouxii eRF3 and GFP were introduced separately. Of these genes, the genes encoding fusion proteins with K. lactis and with D. hansenii were prepared in the same manner as described in Example 6. The gene encoding a fusion protein composed of the prion domain of Z. rouxii eRF3 and GFP was prepared by cloning the Z. rouxii eRF3 prion domain amplified with the following primers into the BamHI-SalI site of pXY112, and then cloning the above-described GFP gene fragment into the SalI-SacI site of this plasmid.

| 5′-CCGGATCCATATGTCTGACCCAAACCAGAA-3′ | (SEQ ID NO: |
|---|---|
| | 31) |
| 5′-GGGGGTCGACATCATTAACGACCCCTTCAT-3′ | (SEQ ID NO: |
| | 32) |

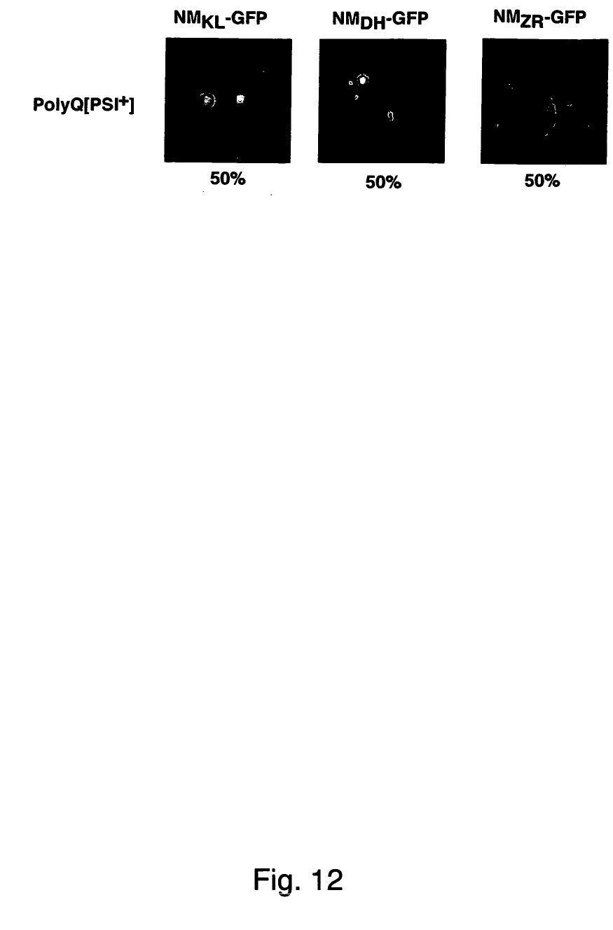

The states of the resultant yeast strains into which the fusion protein genes had been introduced were observed with a fluorescent microscope. The results are shown in FIG. 12. As shown in this Figure, when eRF3 of any yeast species was used, aggregates of the fusion protein were observed, indicating that the [PSI⁺] phenotype was retained.

### EXAMPLE 13

### Confirmation of Contact of Polyglutamine-Replaced eRF3 with Heterologous eRF3

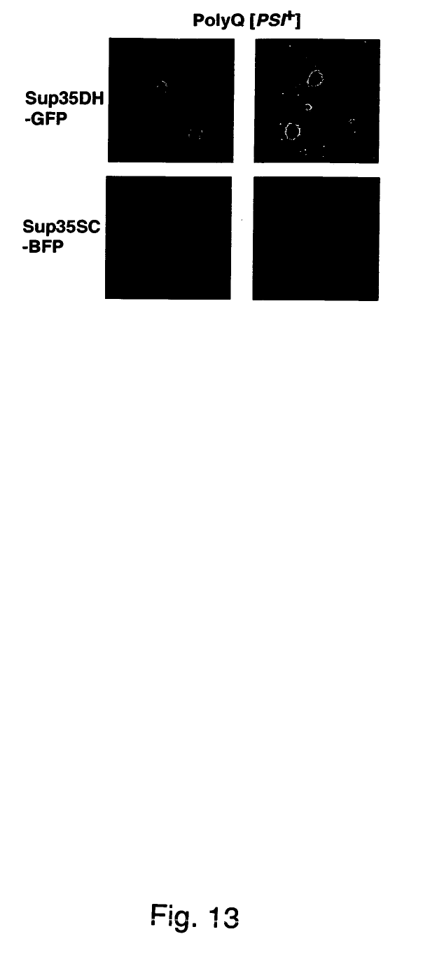

A gene that expresses a fusion protein composed of the above-described polyglutamine-replaced eRF3 and BFP (Poly Q-SC-BFP) was prepared in the same manner as described in Example 7. This gene was introduced into 74-D694Δsup35 strain to render the phenotype of this strain [PSI⁺]. To this yeast strain, a gene that expresses a fusion protein composed of D. hansenii eRF3 and GFP (DH-GFP) was introduced, followed by observation with a fluorescent microscope (FIG. 13). DHGFP gene was prepared by cloning a gene fragment of NMDH-FP amplified by PCR into the SacI site of pRS314.

As shown in FIG. 13, the locations of aggregates of Poly Q-SC-GFP and those of DH-GFP completely coincided with each other. Thus, it is considered that both aggregates are forming co-aggregates.

### EXAMPLE 14

### Assay of the Phenotype after Removal of Polyglutamine-Replaced eRF3

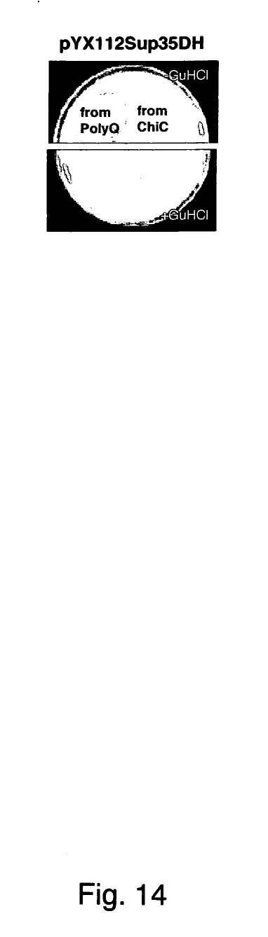

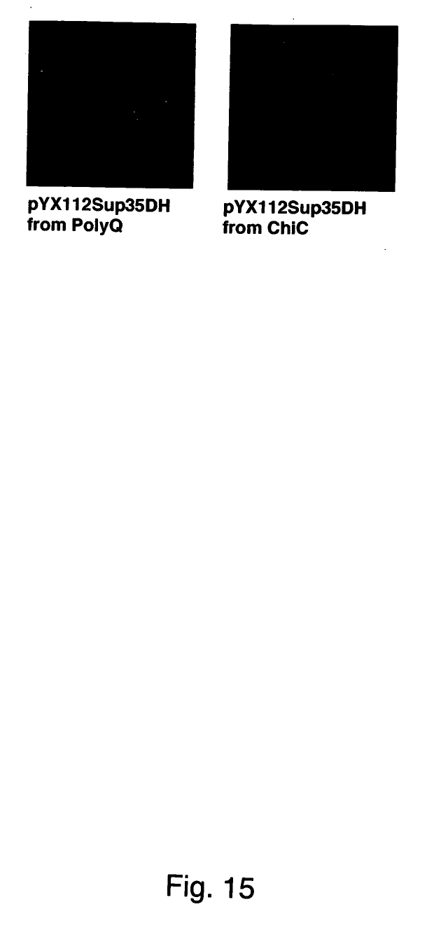

From 74-D694Δsup35 strain into which Poly Q-SC-BFP gene and DH-GFP gene had been introduced, the plasmid comprising Poly Q-SC-BFP gene was removed (by selection of those colonies from which the plasmid had dropped off). Subsequently, the resultant yeast strain was cultured in the same manner as described in Example 3, followed by examination of colony color. This strain was also observed with a fluorescent microscope. The states of colonies are shown in FIG. 14, and fluorescent microphotographs are shown in FIG. 15. The left photograph shows the results when Poly Q-SC-BFP gene was removed. The right photograph shows the results when ChiC gene prepared in Example 5 was introduced and removed instead of Poly Q-SC-BFP gene.

As shown in these Figures, when Poly Q-SC-BFP gene was introduced, the colony color was white and aggregate formation could be observed even after removal of this gene. Thus, [PSI⁺] phenotype was retained. On the contrary, when ChiC gene was introduced, the phenotype changed to [psi] after removal of this gene.

All publications, patents and patent applications cited in the present specification are incorporated herein by reference in their entirety.

### INDUSTRIAL APPLICABILITY

The present invention provides K. lactis- and Y. lipolytica-derived peptides involved in the transmission of prionization between heterologous eRF3 proteins. These peptides are useful in examining the mechanism of transmission of prionization between heterologous eRF3 proteins.

Furthermore, eRF3 proteins comprising the Y. lipolytica-derived peptide undergo prionization at a high frequency, and the manner of transmission of such prionization resembles the manner of transmission of abnormal PrP. Therefore, by using this Y. lipolytica-derived peptide, it becomes possible to screen for substances that inhibit transmission of abnormal PrP or to detect abnormal PrP.

Sequence Listing Free Text

SEQ ID NO: 1 shows the amino acid sequence of a repeat motif contained in K. lactis eRF3.

SEQ ID NO: 2 shows the amino acid sequence of a repeat motif contained in Y. lipolytica eRF3.

SEQ ID NO: 3 shows the nucleotide sequence of a primer used for amplifying C. maltosa eRF3 gene.

SEQ ID NO: 4 shows the nucleotide sequence of a primer used for amplifying D. hansenii eRF3 gene.

SEQ ID NO: 5 shows the nucleotide sequence of a primer used for amplifying K. lactis eRF3 gene.

SEQ ID NO: 6 shows the nucleotide sequence of a primer used for amplifying Y. lipolytica eRF3 gene.

SEQ ID NO: 7 shows the nucleotide sequence of a primer used for amplifying Z. rouxii eRF3 gene.

SEQ ID NO: 8 shows the nucleotide sequence of a primer used for preparing ChiA, a chimeric eRF3.

SEQ ID NO: 9 shows the nucleotide sequence of a primer used for preparing ChiA, a chimeric eRF3.

SEQ ID NO: 10 shows the nucleotide sequence of a primer used for preparing ChiB, a chimeric eRF3.

SEQ ID NO: 11 shows the nucleotide sequence of a primer used for preparing ChiB, a chimeric eRF3.

SEQ ID NO: 12 shows the nucleotide sequence of a primer used for preparing ChiC, a chimeric eRF3.

SEQ ID NO: 13 shows the nucleotide sequence of a primer used for preparing ChiC, a chimeric eRF3.

SEQ ID NO: 14 shows the nucleotide sequence of a primer used for preparing ChiD, a chimeric eRF3.

SEQ ID NO: 15 shows the nucleotide sequence of a primer used for preparing ChiD, a chimeric eRF3.

SEQ ID NO: 16 shows the nucleotide sequence of a primer used for preparing a gene that expresses a fusion protein composed of the prion domain of S. cerevisiae eRF3 and GFP.

SEQ ID NO: 17 shows the nucleotide sequence of a primer used for preparing a gene that expresses a fusion protein composed of the prion domain of S. cerevisiae eRF3 and GFP.

SEQ ID NO: 18 shows the nucleotide sequence of a primer used for preparing a gene that expresses a fusion protein composed of the prion domain of K. lactis eRF3 and GFP.

SEQ ID NO: 19 shows the nucleotide sequence of a primer used for preparing a gene that expresses a fusion protein composed of the prion domain of K. lactis eRF3 and GFP.

SEQ ID NO: 20 shows the nucleotide sequence of a primer used for preparing a gene that expresses a fusion protein composed of the prion domain of D. hansenii eRF3 and GFP.

SEQ ID NO: 21 shows the nucleotide sequence of a primer used for preparing a gene that expresses a fusion protein composed of the prion domain of D. hansenii eRF3 and GFP.

SEQ ID NO: 22 shows the nucleotide sequence of a primer used for preparing a gene that expresses a fusion protein composed of S. cerevisiae eRF3 and BFP.

SEQ ID NO: 23 shows the nucleotide sequence of a primer used for preparing a gene that expresses a fusion protein composed of S. cerevisiae eRF3 and BFP.

SEQ ID NO: 24 shows the nucleotide sequence of a primer used for preparing a gene that expresses a fusion protein composed of K. lactis eRF3 and GFP.

SEQ ID NO: 25 shows the nucleotide sequence of a primer used for preparing a gene that expresses a fusion protein composed of K. lactis eRF3 and GFP.

SEQ ID NO: 26 shows the nucleotide sequence of a primer used for preparing YLRP eRF3.

SEQ ID NO: 27 shows the nucleotide sequence of a primer used for preparing YLRP eRF3.

SEQ ID NO: 28 shows the nucleotide sequence of the PCR template used for preparing polyglutamine-eplaced eRF3.

SEQ ID NO: 29 shows the nucleotide sequence of a primer used for preparing polyglutamine-replaced eRF3.

SEQ ID NO: 30 shows the nucleotide sequence of a primer used for preparing polyglutamine-eplaced eRF3.

SEQ ID NO: 31 shows the nucleotide sequence of a primer used for preparing a gene that expresses a fusion protein composed of Z. rouxii eRF3 and GFP.

SEQ ID NO: 32 shows the nucleotide sequence of a primer used for preparing a gene that expresses a fusion protein composed of Z. rouxii eRF3 and GFP.

1. A peptide represented by the amino acid sequence as shown in SEQ ID NO: 1.

2. A yeast eRF3 in which repetitions of the amino acid sequence as shown in SEQ ID NO: 1 are integrated into its prion domain.

3. A peptide represented by the amino acid sequence as shown in SEQ ID NO: 2.

4. A yeast eRF3 in which repetitions of the amino acid sequence as shown in SEQ ID NO: 2 are integrated into its prion domain.

5. A yeast eRF3 in which a part of the sequence of its prion domain is replaced with a polyglutamine sequence.

6. A method of screening for substances that inhibit transmission of an abnormal PrP, comprising the following steps (1) to (3):
(1) contacting a yeast having a prionized eRF3 with a test substance,
(2) allowing the yeast in step (1) to express a yeast eRF3 having repetitions of the amino acid sequence as shown in SEQ ID NO: 2 in its prion domain, and
(3) determining whether or not the test substance inhibits prionization of the yeast eRF3 expressed in step (2).

7. The method according to claim 6, wherein said yeast eRF3 having repetitions of the amino acid sequence as shown in SEQ ID NO: 2 in its prion domain is YL eRF3, YLRP eRF3 or Δ121YLRP eRF3.

8. The method according to claim 6, wherein said yeast is 74-D694Δsup35 strain, and the step of determining whether or not the test substance inhibits prionization is a step of determining the color of the colonies formed by said yeast.

9. The method according to claim 6, wherein the step of determining whether or not the test substance inhibits prionization is a step of determining whether or not aggregates are formed in cells of said yeast.

10. A method of detecting an abnormal PrP, comprising the following steps (1) and (2):
(1) transferring a test sample into cells of a yeast comprising a yeast eRF3 in which repetitions of the amino acid sequence as shown in SEQ ID NO: 2 or a PrP sequence is integrated into its prion domain, and
(2) determining whether the yeast eRF3 is prionized or not.

11. The method according to claim 10, wherein the yeast eRF3 in which repetitions of the amino acid sequence as shown in SEQ ID NO: 2 or a PrP sequence is integrated into its prion domain is YL eRF3, YLRP eRF3 or Δ121YLRP eRF3.

12. The method according to claim 10, wherein the step of determining whether the yeast eRF3 is prionized or not is a step of determining the color of the colonies formed by said yeast.

13. The method according to claim 10, wherein the step of determining whether the yeast eRF3 is prionized or not is a step of determining whether or not aggregates are formed in cells of said yeast.

14. A method of creating an abnormal PrP, comprising contacting a normal PrP with a yeast eRF3 in which repetitions of the amino acid sequence as shown in SEQ ID NO: 1 are integrated into its prion domain or a yeast eRF3 in which a part of the sequence of its prion domain is replaced with a polyglutamine sequence.

15. The method according to claim 14, wherein the yeast eRF3 in which repetitions of the amino acid sequence as shown in SEQ ID NO: 1 are integrated into its prion domain is KL eRF3, ChiB or ChiC.

16. The method according to claim 14, wherein the yeast eRF3 in which a part of the sequence of its prion domain is replaced with a polyglutamine sequence is a peptide obtainable by replacing the oligopeptide repeat region of Saccharomyces cerevisiae eRF3 with polyglutamine.

17. A method of screening for substances that restore an abnormal PrP to a normal PrP, comprising the following steps (1) to (3):
(1) obtaining an abnormal PrP by contacting a normal PrP with a yeast eRF3 in which repetitions of the amino acid sequence as shown in SEQ ID NO: 1 are integrated into its prion domain or a yeast eRF3 in which a part of the sequence of its prion domain is replaced with a polyglutamine sequence,
(2) contacting the abnormal PrP with a test substance, and
(3) determining whether or not the test substance restores the abnormal PrP to a normal PrP.

18. The method according to claim 17, wherein the yeast eRF3 in which repetitions of the amino acid sequence as shown in SEQ ID NO: 1 are integrated into its prion domain is KL eRF3, ChiB or ChiC.

19. The method according to claim 17, wherein the yeast eRF3 in which a part of the sequence of its prion domain is replaced with a polyglutamine sequence is a peptide obtainable by replacing the oligopeptide repeat region of Saccharomyces cerevisiae eRF3 with polyglutamine.
